(19) United States
(12) Patent Application Publication
(10) Pub. No.: US 2023/0174677 A1
(43) Pub. Date: Jun. 8, 2023

(54) Use of Approved Bispecific EGFR and Met Specific Antibody Drug Product to Treat Non-Small Cell Lung Cancer (NSCLCL)

(71) Applicant: Janssen Biotech, Inc., Horsham, PA (US)

(72) Inventor: Roland Knoblauch, Doylestown, PA (US)

(21) Appl. No.: 17/988,176

(22) Filed: Nov. 16, 2022

Related U.S. Application Data
(60) Provisional application No. 63/279,852, filed on Nov. 16, 2021.

(51) Int. Cl. C07K 16/46; A61P 35/00

(52) U.S. Cl. CPC C07K 16/468; A61P 35/00

(57) ABSTRACT

The present invention relates to methods of treating lung cancer using an approved drug product comprising amivantamab. Also described are drug products containing amivantamab, and methods of selling or offering for sale a drug product comprising amivantamab.

### CROSS-REFERENCE TO RELATED APPLICATIONS

This application claims the benefit of U.S. Provisional Patent Application No. 63/279,852, filed Nov. 16, 2021, the disclosure of which is herein incorporated by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6657USNP1SEQLIST.xml”, creation date of Nov. 14, 2022, and a size of 24 kilobytes (KB). This sequence listing submitted is part of the specification and is herein incorporated by reference in its entirety.

### FIELD OF THE INVENTION

The present invention relates to methods of treating of patients with non-small cell lung cancer (NSCLC) with epidermal growth factor receptor (EGFR) exon 20 insertion mutations using an approved drug product comprising amivantamab. Also described are methods of selling or offering for sale an approved drug product comprising amivantamab for the treatment of NSCLC.

### BACKGROUND OF THE INVENTION

Worldwide, lung cancer is the most commonly diagnosed cancer (11.6% of total cases) and the leading cause of cancer death (18.4% of total cancer deaths) (Globocan 2018: Cancer Fact Sheets: Lung. World Health Organization International Agency for Research on Cancer). Non-Small Cell Lung Cancer (NSCLC) accounts for 80% to 85% of lung cancers (American Cancer Society 2020: About Lung Cancer). Five-year survival rates for NSCLC depend on the stage at diagnosis, ranging from 57% for localized cancer to 5% for cancer that has spread to distant locations (Surveillance, Epidemiology, and End Results Program (SEER). SEER Stat Fact Sheets: Lung and Bronchus Cancer). In patients with metastatic NSCLC, clinical guidelines recommend testing for activating mutations (National Comprehensive Cancer Network (NCCN). Non-Small Cell Lung Cancer (Version 1.2020); Planchard D et al. ESMO Guidelines Committee. Metastatic non-small cell lung cancer: ESMO Clinical Practice Guidelines for diagnosis, treatment and follow-up. Ann Oncol. 2018; 29(Suppl4)). Among patients with NSCLC, the most prevalent actionable driver mutations are those that result in the activation of EGFR (Skoulidis F, Heymach J V. Co-occurring genomic alterations in non-small-cell lung cancer biology and therapy. Nat Rev Cancer. 2019; 19(9):495-509; Devarakonda S, Morgensztern D, Govindan R. Genomic alterations in lung adenocarcinoma. Lancet Oncol 2015; 16(7):e342-351; Jánne P A, Johnson B E. Effect of Epidermal Growth Factor Receptor Tyrosine Kinase Domain Mutations on the Outcome of Patients with Non-Small Cell Lung Cancer Treated with Epidermal Growth Factor Receptor Tyrosine Kinase Inhibitors. Clin Cancer Res. 2006; 12(14):4416s-4420s).

Activating EGFR mutations are significantly more common in certain groups of patients, including East Asians (up to ˜50%), women, and non-smokers (Janne 2006). Several approved drugs, including tyrosine kinase inhibitors (TKIs) erlotinib and afatinib, are the standard of care first-line therapy for NSCLC patients with tumors bearing specific EGFR mutations. Although the majority of these patients initially respond, virtually all acquire resistance, preventing a durable response. Moreover, tumors arising from EGFR exon 20 insertion mutations (Exon20ins) are known to be insensitive to treatment with approved EGFR TKIs, and these patients remain a population of unmet medical need (Arcila M E, et al. EGFR exon 20 insertion mutations in lung adenocarcinomas: prevalence, molecular heterogeneity, and clinicopathologic characteristics. Mol Cancer Ther. 2013; 12(2):220-229).

Amivantamab (JNJ-61186372) is a fully human IgG1-based bispecific antibody directed against the EGF and cMet receptors. By inhibiting EGFR and cMet signaling functions, amivantamab may disrupt these signaling pathways, thereby preventing tumor growth and progression. Furthermore, the presence of high levels of EGFR and cMet on the surface of tumor cells allows for targeting of these cells for destruction by immune effector cells, through antibody-dependent cellular cytotoxicity (ADCC) and antibody-dependent cellular phagocytosis (ADCP) mechanisms.

Currently recognized treatments for NSCLC with Exon20ins include various chemotherapeutic agents. However, the disease remains largely incurable. Thus, there is a need for additional therapeutics for NSCLC with Exon20ins.

### SUMMARY OF THE INVENTION

The invention provides a method of treating non-small cell lung cancer (NSCLC) comprising administering an approved drug product comprising amivantamab to an adult patient with NSCLC in an amount that is described in a drug product label for the drug product. In some embodiments the NSCLC is locally advanced or metastatic. In some embodiments the NSCLC has an at least one epidermal growth factor receptor (EGFR) exon 20 insertion mutation. In some embodiments the patient was previously administered a platinum-based chemotherapy drug. In some embodiments the platinum-based chemotherapy drug is selected from the group consisting of cisplatin, carboplatin, oxaliplatin, nedaplatin, lobaplatin and heptaplatin.

In some embodiments the administration of amivantamab provides an improvement in overall response, wherein the overall response is measured according to Response Evaluation Criteria in Solid Tumors (RECIST v1.1) of the adult patient with NSCLC.

In some embodiments the amivantamab is administered intravenously (IV) at a dose of about 1050 mg. In some embodiments the amivantamab is administered intravenously (IV) at a dose of about 1400 mg.

In some embodiments the dose is administered weekly for 4 weeks, followed by administration every two weeks. In some embodiments the dose administered during the first week is a split dose. In some embodiments the split dose is administered in two or more infusions over a period of no more than 3 days.

The invention also provides a method of selling an approved drug product comprising amivantamab, the method comprising selling such drug product, wherein a drug product label for a reference listed drug for such drug product includes instructions for treating NSCLC.

In some embodiments the drug product is a biosimilar drug product, a Biologic License Application drug product or a supplemental Biologic License Application drug product.

The invention also provides a method of offering for sale an approved drug product comprising amivantamab, the method comprising offering for sale such drug product, wherein a drug product label for a reference listed drug for such drug product includes instructions for treating NSCLC.

The invention also provides a method of selling an approved drug product comprising amivantamab, the method comprising selling such drug product, wherein the drug product label for a reference listed drug for such drug product comprises overall response rate (ORR) data) according to Response Evaluation Criteria in Solid Tumors (RECIST v1.1).

The invention also provides a method of offering for sale an approved drug product comprising amivantamab, said method comprising offering for sale such drug product, wherein the drug product label for a reference listed drug for such drug product comprises overall response rate (ORR) data) according to Response Evaluation Criteria in Solid Tumors (RECIST v1.1).

The invention also provides a method of improving overall response in an adult patient with NSCLC, wherein the overall response is measured according to Response Evaluation Criteria in Solid Tumors (RECIST v1.1), the method comprising administering to the adult patient an approved drug product comprising amivantamab.

In some embodiments the approved drug product is a biosimilar drug product, a Biologic License Application drug product or a supplemental Biologic License Application drug product.

In some embodiments the NSCLC is locally advanced or metastatic. In some embodiments the NSCLC has an at least one epidermal growth factor receptor (EGFR) exon 20 insertion mutation.

In some embodiments the patient was previously administered a platinum-based chemotherapy drug.

In some embodiments the approved drug product provides an improvement in the overall response, wherein the overall response is either complete response (CR) or partial response (PR) as measured according to RECIST v1.1.

In some embodiments a drug product label for a reference listed drug for such approved drug product comprises overall response rate (ORR) data according to Response Evaluation Criteria in Solid Tumors (RECIST v1.1).

In some embodiments a drug product label for a reference listed drug for such approved drug product includes instructions for treating NSCLC.

In some embodiments the method further comprises selling such approved drug product, wherein a drug product label for a reference listed drug for such approved drug product includes instructions for treating NSCLC.

In some embodiments the drug product label comprises overall response rate (ORR) data.

The invention also provides a method of improving overall response in an adult patient with NSCLC, said method comprising providing to said patient an approved drug product comprising amivantamab. In some embodiments the NSCLC is locally advanced or metastatic. In some embodiments the NSCLC has an at least one epidermal growth factor receptor (EGFR) exon 20 insertion mutation.

In some embodiments the patient was previously administered a platinum-based chemotherapy drug.

In some embodiments a drug product label for a reference listed drug for such approved drug product comprises overall response rate (ORR) data.

In some embodiments a drug product label for a reference listed drug for such approved drug product includes instructions for treating NSCLC.

### DETAILED DESCRIPTION OF THE INVENTION

“RYBREVANT™” comprises amivantamab as an active ingredient. RYBREVANT injection is a sterile, preservative free, colorless to pale yellow solution for intravenous infusion. Each single dose vial contains 350 mg/7 mL (50 mg/mL) of amivantamab.

“EGFR” or “Epidermal growth factor receptor” refers to the human EGFR (also known as HER1 or ErbB1 (Ullrich et al., Nature 309:418-425, 1984) having the amino acid sequence shown in SEQ ID NO: 21 and in GenBank accession number NP_005219, as well as naturally occurring variants thereof. Such variants include the well-known EGFRvIII and other alternatively spliced variants (e.g., as identified by SwissProt Accession numbers P00533-1 (wild type; identical to SEQ ID NO: 21 and NP_005219), P00533-2 (F404L/L4055), P00533-3 (628-705: CTGPGLEGCP . . . GEAPNQALLR→PGNESLKAML . . . SVIITASSCH and 706-1210 deleted), P00533-4 (C628S and 629-1210 deleted), variants GlnQ98, R266, K521, 1674, G962, and P988 (Livingston et al., NIEHS-SNPs, environmental genome project, NIEHS ES15478), T790M, L858R/T790M and del(E746, A750).

SEQ ID NO: 21, Homo sapiens, EGFR (includes signal sequence of 24 aa. Mature protein starts at residue 25)

| | |
|---|---|
| 1 | MRPSGTAGAA LLALLAALCP ASRALEEKKV CQGTSNKLTQ LGTFEDHFLS LQRMFNNCEV |
| 61 | VLGNLEITYV QRNYDLSFLK TIQEVAGYVL IALNTVERIP LENLQIIRGN MYYENSYALA |
| 121 | VLSNYDANKT GLKELPMRNL QEILHGAVRF SNNPALCNVE SIQWRDIVSS DFLSNMSMDF |
| 181 | QNHLGSCQKC DPSCPNGSCW GAGEENCQKL TKIICAQQCS GRCRGKSPSD CCHNQCAAGC |
| 241 | TGPRESDCLV CRKFRDEATC KDTCPPLMLY NPTTYQMDVN PEGKYSFGAT CVKKCPRNYV |
| 301 | VTDHGSCVRA CGADSYEMEE DGVRKCKKCE GPCRKVCNGI GIGEFKDSLS INATNIKHFK |
| 361 | NCTSISGDLH ILPVAFRGDS FTHTPPLDPQ ELDILKTVKE ITGFLLIQAW PENRTDLHAF |
| 421 | ENLEIIRGRT KQHGQFSLAV VSLNITSLGL RSLKEISDGD VIISGNKNLC YANTINWKKL |
| 481 | FGTSGQKTKI ISNRGENSCK ATGQVCHALC SPEGCWGPEP RDCVSCRNVS RGRECVDKCN |
| 541 | LLEGEPREFV ENSECIQCHP ECLPQAMNIT CTGRGPDNCI QCAHYIDGPH CVKTCPAGVM |
| 601 | GENNTLVWKY ADAGHVCHLC HPNCTYGCTG PGLEGCPTNG PKIPSIATGM VGALLLLLVV |
| 661 | ALGIGLFMRR RHIVRKRTLR RLLQERELVE PLTPSGEAPN QALLRILKET EFKKIKVLGS |
| 721 | GAFGTVYKGL WIPEGEKVKI PVAIKELREA TSPKANKEIL DEAYVMASVD NPHVCRLLGI |
| 781 | CLTSTVQLIT QLMPFGCLLD YVREHKDNIG SQYLLNWCVQ IAKGMNYLED RRLVHRDLAA |
| 841 | RNVLVKTPQH VKITDFGLAK LLGAEEKEYH AEGGKVPIKW MALESILHRI YTHQSDVWSY |
| 901 | GVTVWELMTF GSKPYDGIPA SEISSILEKG ERLPQPPICT IDVYMIMVKC WMIDADSRPK |
| 961 | FRELIIEFSK MARDPQRYLV IQGDERMHLP SPTDSNFYRA LMDEEDMDDV VDADEYLIPQ |
| 1021 | QGFFSSPSTS RTPLLSSLSA TSNNSTVACI DRNGLQSCPI KEDSFLQRYS SDPTGALTED |
| 1081 | SIDDTFLPVP EYINQSVPKR PAGSVQNPVY HNQPLNPAPS RDPHYQDPHS TAVGNPEYLN |
| 1141 | TVQPTCVNST FDSPAHWAQK GSHQISLDNP DYQQDFFPKE AKPNGIFKGS TAENAEYLRV |
| 1201 | APQSSEFIGA |

“EGFR exon 20 insertion mutation” or “EGFR exon 20 mutations” or “EGFR Exon20ins” or “Exon20ins” refer to the human EGFR gene encompassing at least one mutation in nucleotides that translate into amino acids at position 762-823, and include a C-helix (762-766) followed by a loop (767-775) (see Yasuda H et al., Science Translational Medicine 2013; 5(216):216ra177 doi 10.1126/scitranslmed.3007205). The insertion mutations of one to seven amino acids in exon 20 form a wedge at the end of the C-helix in EGFR that promotes active kinase conformation. An EGFR exon 20 insertion mutation can be detected using known methods, such as sequencing, including next-generation sequencing (NGS), fluorescent in situ hybridization, immunohistochemistry, flow cytometry or western blotting.

“MET” or “c-Met” or “Hepatocyte growth factor receptor” as used herein refers to the human c-Met having the amino acid sequence shown in SEQ ID NO: 22 or as identified by SwissProt Accession number P08581 and natural variants thereof.

```
SEQ ID NO: 22
      1 mkapavlapg ilvllftlvq rsngeckeal aksemnvnmk yqlpnftaet piqnvilheh
    61 hiflgatnyi yvlneedlqk vaeyktgpvl ehpdcfpcqd csskanlsgg vwkdninmal
  121 vvdtyyddql iscgsvnrgt cqrhvfphnh tadiqsevhc ifspqieeps qcpdcvvsal
  181 gakvlssvkd rfinffvgnt inssyfpdhp ihsisvrrlk etkdgfmflt dqsyidvlpe
  241 frdsypikyv hafesnnfiy fltvqretld aqtfhtriir fcsinsglhs ymemplecil
  301 tekrkkrstk kevfnilqaa yvskpgaqla rqigaslndd ilfgvfaqsk pdsaepmdrs
  361 amcafpikyv ndffnkivnk nnvrclqhfy gpnhehcfnr tllrnssgce arrdeyrtef
  421 ttalqrvdlf mgqfsevllt sistfikgdl tianlgtseg rfmqvvvsrs gpstphvnfl
  481 idshpvspev ivehtlnqng ytlvitgkki tkiplnglgc rhfqscsqcl sappfvqcgw
  541 chdkcvrsee clsgtwtqqi clpaiykvfp nsapleggtr iticgwdfgf rrnnkfdlkk
  601 trvllgnesc tltlsestmn tlkctvgpam nkhfnmsiii snghgttqys tfsyvdpvit
  661 sispkygpma ggtlltltgn ylnsgnsrhi siggktctlk svsnsilecy tpaqtistef
  721 avklkidlan retsifsyre dpivyeihpt ksfistwwke plnivsflfc fasggstitg
  781 vgknlnsvsv prmvinvhea grnftvacqh rsnseiicct tpslqqlnlq lplktkaffm
  841 idgilskyfd liyvhnpvfk pfekpvmism gnenvieikg ndidpeavkg evlkvgnksc
  901 enihlhseav lctvpndllk Inselniewk qaisstvigk vivqpdqnft gliagvvsis
  961 talllllgff iwlkkrkqik dlgselvryd arvhtphldr ivsarsvspt temvsnesvd
1021 yratfpedqf pnssqngscr qvqypltdms piltsgdsdi sspllqntvh idlsalnpel
1081 vqavqhvvig psslivhfne vigrghfgcv yhgtlldndg kkihcavksl nritdigevs
1141 qfltegiimk dfshpnvlsl lgiclrsegs plvvlpymkh gdlrnfirne thnptvkdli
1201 gfglqvakgm kylaskkfvh rdlaarncml dekftvkvad fglardmydk eyysvhnktg
1261 aklpvkwmal eslqtqkftt ksdvwsfgvl lwelmtrgap pypdvntfdi tvyllqgrrl
1321 iqpeycpdpl yevmlkcwhp kaemrpsfse ivsrisaifs tfigehyvhv natyvnvkcv
1381 apypsllsse dnaddevdtr pasfwets
```

“EGFR and/or c-Met expressing cancer” refers to cancer that has detectable expression of EGFR and/or c-Met or has EGFR or c-Met mutation or amplification. EGFR or c-Met expression, amplification and mutation status can be detected using know methods, such as sequencing, including next-generation sequencing, fluorescent in situ hybridization, immunohistochemistry, flow cytometry or western blotting.

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J Exp Med 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol Biol 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

A “chemotherapy” as used herein, refers to a chemotherapeutic agent, as defined above, or a combination of two, three or four chemotherapeutic agents, for the treatment of cancer. When a chemotherapy consists of more than one chemotherapeutic agents, the chemotherapeutic agents can be administered to the patient on the same day or on different days in the same treatment cycle.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, lobaplatin, heptaplatin, gemcitabine in combination with cisplatin, carboplatin in combination with pemetremed.

“Drug product” or “approved drug product” is product that contains an active pharmaceutical ingredient that has been approved for marketing for at least one indication by a governmental authority, e.g., the Food and Drug Administration or the similar authority in other countries. In the present invention, RYBREVANT is an approved drug product. The approved drug product could also be a biosimilar amivantamab drug product, a Biologic License Application drug product or a supplemental Biologic License Application drug product.

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A multispecific antibody, such as a bispecific antibody or a trispecific antibody is included in the term monoclonal antibody.

“Recombinant” includes antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“In combination with” means that two or more therapeutics are administered to a subject together in a mixture, concurrently as single agents or sequentially as single agents in any order.

“Pharmaceutical composition” refers to a product that results from combining an anti-EGFR/anti-MET antibody and a pharmaceutically acceptable carrier.

“Treat” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or disease as well as those subjects prone to have the physiological change or disease.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body and, in the case of patients with locally advanced or metastatic NSCLC with EGFR exon 20 insertion mutations, a confirmed overall response rate (ORR) according to Response Evaluation Criteria in Solid Tumors (RECIST v1.1).

“RECIST” or “Response Evaluation Criteria in Solid Tumors” version 1.1 are criteria developed to facilitate objective assessment of change in tumor burden such as tumor shrinkage and time to development of disease progression. RECIST guidelines are posted on the worldwide web at http://ctep(dot)cancer(dot)gov(slash)protocoldevelopment(slash)docs(slash)recist_guideline(dot)pdf.

“Complete Response” or “CR” is both (1) disappearance of all target lesions and (2) any pathological lymph nodes (whether target or non-target) must have reduction in short axis to <10 mm.

“Partial Response” or “PR” is at least a 30% decrease in the sum of diameters of target lesions, taking as reference the baseline sum diameters.

“Target lesion” is a lesion selected as a representative lesion to be measured, when more than one lesion is present in a subject.

“Overall response” is the response to treatment recorded from the start of the study treatment until the end of treatment. “Improved overall response” is the overall response when either partial or complete response to therapy is achieved.

“Overall response rate” or “ORR” is the proportion of patients who have a partial or complete response to therapy.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

### Pharmaceutical Compositions

The invention provides a pharmaceutical composition comprising a bispecific anti-EGFR/anti-MET antibody.

The pharmaceutical composition is useful for intravenous administration of the bispecific anti-EGFR/anti-MET antibody to a subject in need of anti-EGFR/anti-MET antibody therapy, such as a subject having a cancer, for example a EGFR exon 20 insertion mutation-positive NSCLC.

In some embodiments, the bispecific anti-EGFR/anti-MET antibody in the pharmaceutical composition comprises a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively; the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively.

In some embodiments, the bispecific anti-EGFR/anti-MET antibody in the pharmaceutical composition comprises a HC1 variable region comprising the amino acid sequence of SEQ ID NO:13 and a LC1 variable region comprising the amino acid sequence of SEQ ID NO:14.

In some embodiments, the bispecific anti-EGFR/anti-MET antibody in the pharmaceutical composition comprises a HC2 variable region comprising the amino acid sequence of SEQ ID NO:15 and a LC1 variable region comprising the amino acid sequence of SEQ ID NO:16.

In some embodiments, the bispecific anti-EGFR/anti-MET antibody in the pharmaceutical composition comprises a HC1 comprising the amino acid sequence of SEQ ID NO:17 and a LC1 comprising the amino acid sequence of SEQ ID NO: 18.

In some embodiments, the bispecific anti-EGFR/anti-MET antibody in the pharmaceutical composition comprises a HC2 comprising the amino acid sequence of SEQ ID NO:19 and a LC2 comprising the amino acid sequence of SEQ ID NO: 20.

EGFR binding arm
>SEQ ID NO: 1 (HCDR1, EGFR binding arm):
TYGMH
>SEQ ID NO: 2 (HCDR2, EGFR binding arm):
VIWDDGSYKYYGDSVKG
>SEQ ID NO: 3 (HCDR3, EGFR binding arm):
DGITMVRGVMKDYFDY
>SEQ ID NO: 4 (LCDR1, EGFR binding arm):
RASQDISSALV
>SEQ ID NO: 5 (LCDR2, EGFR binding arm):
DASSLES
>SEQ ID NO: 6 (LCDR3, EGFR binding arm):
QQFNSYPLT
>SEQ ID NO: 7 (HCDR1, c-Met binding arm):
SYGIS
>SEQ ID NO: 8 (HCDR2, c-Met binding arm):
WISAYNGYTNYAQKLQG
>SEQ ID NO: 9 (HCDR3, c-Met binding arm):
DLRGTNYFDY
>SEQ ID NO: 10 (LCDR1, c-Met binding arm):
RASQGISNWLA
>SEQ ID NO: 11 (LCDR2, c-Met binding arm):
AASSLLS
>SEQ ID NO: 12 (LCDR3, c-Met binding arm):
QQANSFPIT
>SEQ ID NO: 13 (VH, EGFR binding arm):
QVQLVESGGGVVQPGRSLRLSCAASGFTFSTYGMHWVRQAPGKGLEWVAVIWDDGSYK
YYGDSVKGRFTISRDNSKNTLYLQMNSLRAEDTAVYYCARDGITMVRGVMKDYFDYWGQGTL
VTVSS
>SEQ ID NO: 14 (VL, EGFR binding arm):
AIQLTQSPSSLSASVGDRVTITCRASQDISSALVWYQQKPGKAPKLLIYDASSLESGVPSRFS
GSESGTDFTLTISSLQPEDFATYYCQQFNSYPLTFGGGTKVEIK
>SEQ ID NO: 15 (VH, c-Met binding arm):
QVQLVQSGAEVKKPGASVKVSCETSGYTFTSYGISWVRQAPGHGLEWMGWISAYNGYTN
YAQKLQGRVTMTTDTSTSTAYMELRSLRSDDTAVYYCARDLRGTNYFDYWGQGTLVTVSS
>SEQ ID NO: 16 (VL, c-Met binding arm):
DIQMTQSPSSVSASVGDRVTITCRASQGISNWLAWFQHKPGKAPKLLIYAASSLLSGVPSRF
SGSGSGTDFTLTISSLQPEDFATYYCQQANSFPITFGQGTRLEIK
>SEQ ID NO: 17 HC1:
QVQLVESGGGVVQPGRSLRLSCAASGFTFSTYGMHWVRQAPGKGLEWVAVIWDDGSYK
YYGDSVKGRFTISRDNSKNTLYLQMNSLRAEDTAVYYCARDGITMVRGVMKDYFDYWGQGTL
VTVSSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFLLYSKLTVDKSRWQQGNVFSCSVMHEALHN
HYTQKSLSLSPGK
>SEQ ID NO: 18 LC1:
AIQLTQSPSSLSASVGDRVTITCRASQDISSALVWYQQKPGKAPKLLIYDASSLESGVPSRFS
GSESGTDFTLTISSLQPEDFATYYCQQFNSYPLTFGGGTKVEIKRTVAAPSVFIFPPSDEQLKSGTA
SVVCLLNNFYPREAKVQWKVDNALQSGNSQESVTEQDSKDSTYSLSSTLTLSKADYEKHKVYA
CEVTHQGLSSPVTKSFNRGEC
>SEQ ID NO: 19 HC2:
QVQLVQSGAEVKKPGASVKVSCETSGYTFTSYGISWVRQAPGHGLEWMGWISAYNGYTN
YAQKLQGRVTMTTDTSTSTAYMELRSLRSDDTAVYYCARDLRGTNYFDYWGQGTLVTVSSAST
KGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVKGFYPSDIA
VEWESNGQPENNYKTTPPVLDSDGSFFLYSRLTVDKSRWQQGNVFSCSVMHEALHNHYTQKSL
SLSPGK
>SEQ ID NO: 20 LC2:
DIQMTQSPSSVSASVGDRVTITCRASQGISNWLAWFQHKPGKAPKLLIYAASSLLSGVPSRF
SGSGSGTDFTLTISSLQPEDFATYYCQQANSFPITFGQGTRLEIKRTVAAPSVFIFPPSDEQLKSGTA
SVVCLLNNFYPREAKVQWKVDNALQSGNSQESVTEQDSKDSTYSLSSTLTLSKADYEKHKVYA

The invention also provides a pharmaceutical composition comprising a bispecific anti-EGFR/anti-MET antibody comprising the VH1 of SEQ ID NO: 13 and the VL1 of SEQ ID NO: 14 and VH2 of SEQ ID NO: 15 and the VL2 of SEQ ID NO: 16, wherein the anti-EGFR/anti-MET antibody concentration in the pharmaceutical composition is about 50 mg/mL.

The invention also provides a pharmaceutical composition comprising a bispecific anti-EGFR/anti-MET antibody comprising the VH1 comprises a HCDR1, the HCDR2 and a HCDR3 amino acid sequences of SEQ ID NOs: 1, 2, and 3, respectively; the VL1 comprises the LCDR1, a LCDR2 and a LCDR3 amino acid sequences of SEQ ID NOs: 4, 5 and 6, respectively; the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively, and wherein the anti-EGFR/anti-MET antibody concentration in the pharmaceutical composition is about 50 mg/mL

The pharmaceutical compositions of the invention further comprise a pharmaceutically acceptable carrier. Exemplary pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

The invention also provides a pharmaceutical composition comprising about 50 mg/mL of the anti-EGFR/anti-MET antibody comprising the VH1 of SEQ ID NO: 13 and the VL1 of SEQ ID NO: 14 and VH2 of SEQ ID NO: 15 and the VL2 of SEQ ID NO: 16 in about EDTA disodium salt dihydrate (0.14 mg), L-histidine (2.3 mg), L-histidine hydrochloride monohydrate (8.6 mg), L-methionine (7 mg), polysorbate 80 (4.2 mg), sucrose (595 mg), and water for injection, USP in a final volume of 7 mL.

The formulations to be used for in vivo administration are generally sterile. Sterility may be readily accomplished, e.g., by filtration through sterile filtration membranes.

### Methods of Treatment

The invention provides methods of treating non small cell lung cancer (NSCLC) with epidermal growth factor receptor (EGFR) exon 20 insertion mutations with an approved drug product.

The anti-EGFR/anti-MET bispecific antibody used in the methods of treatment of the invention is amivantamab. Amivantamab comprises the VH1 comprising a HCDR1, the HCDR2 and a HCDR3 amino acid sequences of SEQ ID NOs: 1, 2, and 3, respectively; the VL1 comprising the LCDR1, a LCDR2 and a LCDR3 amino acid sequences of SEQ ID NOs: 4, 5 and 6, respectively; the VH2 comprising the HCDR1, the HCDR2 and the HCDR3 amino acid sequences of SEQ ID NOs: 7, 8 and 9, respectively; and the VL2 comprising the LCDR1, the LCDR2 and the LCDR3 amino acid sequences of SEQ ID NOs: 10, 11 and 12, respectively. Amivantamab comprises the VH1 of SEQ ID NO: 13 and the VL1 of SEQ ID NO: 14 and VH2 of SEQ ID NO: 15 and the VL2 of SEQ ID NO: 16. Amivantamab HC1 amino acid sequence is shown in SEQ ID NO: 17 and LC1 amino acid sequence is shown in SEQ ID NO: 18; the HC2 amino acid sequence is shown in SEQ ID NO: 19 and LC2 amino acid sequence is shown in SEQ ID NO: 20. Amivantamab was described in U.S. Pat. No. 9,593,164.

The invention also provides a method of treating NSCLC with EGFR exon 20 insertion mutations, comprising administering to a subject in need thereof an approved drug product comprising the anti-EGFR/anti-MET bispecific antibody comprising the VH1 of SEQ ID NO: 13 and the VL1 of SEQ ID NO: 14 and VH2 of SEQ ID NO: 15 and the VL2 of SEQ ID NO: 16 intravenously for a time sufficient to treat the subject, wherein the anti-EGFR/anti-MET antibody concentration in the approved drug product is about 50 mg/mL.

The invention also provides a method of treating NSCLC with EGFR exon 20 insertion mutations, comprising administering to a subject in need thereof an approved drug product comprising the anti-EGFR/anti-MET bispecific antibody comprising the VH1 of SEQ ID NO: 13 and the VL1 of SEQ ID NO: 14 and VH2 of SEQ ID NO: 15 and the VL2 of SEQ ID NO: 16 in about 10 mM L-histidine, about 8.5% (w/v) sucrose, about 0.06% (w/v) polysorbate 80 (PS80), about 1 mg/mL methionine, about 20 μg/mL EDTA, about pH 5.7 for a time sufficient to treat the subject.

The anti-EGFR/anti-MET bispecific antibodies in the pharmaceutical compositions of the invention may induce killing of EGFR-expressing and/or MET-expressing tumor cells by antibody-dependent cell-mediated cytotoxicity (ADCC) and/or trogocytosis. In some embodiments, the anti-EGFR/anti-MET bispecific antibodies in the pharmaceutical compositions of the invention may disrupt EGFR and MET signaling functions through blocking ligand binding and/or degrading EGFR and/or MET.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells. For example, NK cells express FcγRIIIa, whereas monocytes express FcγRI, FcγRII and FcγRIIIa. Death of the antibody-coated target cell, such as EGFR-expressing and/or MET-expressing cells, occurs as a result of effector cell activity through the secretion of membrane pore-forming proteins and proteases. To assess ADCC activity of an antibody that specifically binds EGFR or MET, the antibody may be added to EGFR and/or MET-expressing cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include lung cancer cells expressing EGFR and/or MET. In an exemplary assay, target cells are pre-labeled with BATDA (PerkinElmer™) for 20 minutes at 37° C., washed and resuspended in DMEM, 10% heat-inactivated FBS, 2 mM L-glutamine (Invitrogen™). Target (1×104 cells) and effector cells (0.5×106 cells) are combined and 100 μl of cells are added per wells. An additional 100 μl are added with or without wild type and protease-resistant mAb constructs. The plates are centrifuged at 200 g for 3 minutes, incubated at 37° C. for 2 hours, and then centrifuged again at 200 g for 3 minutes. A total of 20 μl of supernatant are removed per well and cell lysis is measured by the addition of 200 μl of the DELPHIA™ Europium-based reagent (PerkinElmer). Fluorescence is measured using an Envision 2101 Multilabel Reader (PerkinElmer™). Data are normalized to maximal cytotoxicity with 0.67% Triton X-100 (Sigma Aldrich) and minimal control determined by spontaneous release of BATDA from target cells in the absence of any antibody using the following equation: (experimental release−spontaneous release)/(maximal release−spontaneous release)×100%.

“Trogocytosis” refers to a process characterized by the transfer of a portion of a cell membrane from a donor cell to an acceptor cell. Typical acceptor cells include macrophages and monocytes. Additional acceptor cells include NK cells, dendritic cells, T cells, B cells and neutrophils. Trogocytosis-mediated transfer of a portion of a cell membrane may include transfer of membrane proteins, for example such as EGFR or c-Met, or antibody-antigen complexes where an antibody is bound to the cell surface molecule. Antibody-mediated trogocytosis may occur via binding to the Fc portion of the antibody to the Fcγ receptor (FcγR) expressed on acceptor cells.

### Patient Selection

Selection of patients for treatment with the pharmaceutical composition of the invention may be based on the presence of EGFR exon 20 insertion mutations. The presence of at least one EGFR exon 20 insertion mutation can be determined using an FDA approved test. The information on FDA approved tests may be available at: www_fda.gov/CompanionDiagnostics.

### Administration

In some embodiments the pharmaceutical composition of the invention may be administered in a total volume of about 21 mL. In some embodiments the pharmaceutical composition of the invention may be administered in a total volume of about 28 mL.

The pharmaceutical compositions of the invention may be administered weekly for 4 weeks, with the initial dose as a split infusion in Week 1 on Day 1 and Day 2, then administered every 2 weeks thereafter until disease progression or unacceptable toxicity. In some embodiments the initial dose provided as a split infusion (“split dose”). The split dose is provided in multiple individual infusions, over a specified period of time, such as no more than 3 days. In some embodiments the split dose is provided in 2 infusions over the period of 2 days. In some embodiments each of the 2 infusions in the split dose is provided once per day.

In some embodiments premedications may optionally be administered prior to the administration of the pharmaceutical compositions of the invention. In some embodiments the premedication is selected from the group comprising an antihistamine, an antipyretic, and a glucocorticoid. In some embodiments the premedication comprises an antihistamine and an antipyretic. In some embodiments the premedication comprises an antihistamine, an antipyretic, and a glucocorticoid. In some embodiments the antihistamine is diphenhydramine. In some embodiments the antihistamine is diphenhydramine equivalent. In some embodiments the antipyretic is acetaminophen. In some embodiments the antipyretic is acetaminophen equivalent. In some embodiments the glucocorticoid is dexamethasone. In some embodiments the glucocorticoid is dexamethasone equivalent. In some embodiments the glucocorticoid is methylprednisolone. In some embodiments the glucocorticoid is methylprednisolone equivalent. In some embodiments the premedication is diphenhydramine and acetaminophen. In some embodiments the premedication is diphenhydramine, acetaminophen, and dexamethasone. In some embodiments the premedication is diphenhydramine, acetaminophen, and methylprednisolone.

In some embodiments the diphenhydramine is administered at a dose of about 25 to 50 mg. In some embodiments the acetaminophen is administered at a dose of about 650 to 1000 mg. In some embodiments the dexamethasone is administered at a dose of about 10 mg. In some embodiments the methylprednisolone is administered at a dose of about 40 mg.

In some embodiments the premedication is administered on the same day and prior in time relative to the administration to the subject of the pharmaceutical composition of the invention. In some embodiments the premedication is administered 15 to 30 minutes prior to the administration of the pharmaceutical composition of the invention. In some embodiments the premedication is administered 30 to 60 minutes prior to the administration of the pharmaceutical composition of the invention. In some embodiments the premedication is administered 45 to 60 minutes prior to the administration of the pharmaceutical composition of the invention.

The pharmaceutical composition of the invention may be administered intravenously. Intravenous administration may be accomplished using an infusion set fitted with a flow regulator and with an in line, sterile, non pyrogenic, low protein binding polyethersulfone (PES) filter (pore size 0.2 micrometer) primed with diluent.

While having described the invention in general terms, the embodiments of the invention will be further disclosed in the following examples that should not be construed as limiting the scope of the claims.

### Embodiments

Embodiment 1. A method of treating non-small cell lung cancer (NSCLC) comprising administering an approved drug product comprising amivantamab to an adult patient with NSCLC in an amount that is described in a drug product label for the drug product.
Embodiment 2. The method of Embodiment 1, wherein the NSCLC is locally advanced or metastatic.
Embodiment 3. The method of Embodiment 1, wherein the NSCLC has an at least one epidermal growth factor receptor (EGFR) exon 20 insertion mutation.
Embodiment 4. The method of Embodiment 1, wherein the patient was previously administered a platinum-based chemotherapy drug.
Embodiment 4a. The method of Embodiment 4, wherein the platinum-based chemotherapy drug is selected from the group consisting of cisplatin, carboplatin, oxaliplatin, nedaplatin, lobaplatin and heptaplatin.
Embodiment 4b. The method of Embodiment 4, wherein the NSCLC has progressed on or after platinum based-chemotherapy.
Embodiment 5. The method of any one of Embodiments 2 to 4, wherein administration of amivantamab provides an improvement in overall response, wherein the overall response is measured according to Response Evaluation Criteria in Solid Tumors (RECIST v1.1) of the adult patient with NSCLC.
Embodiment 5a. The method of Embodiment 5, wherein the overall response is either complete response (CR) or partial response (PR), where in CR or PR are measured according to Response Evaluation Criteria in Solid Tumors (RECIST v1.1)
Embodiment 6. The method of any one of Embodiments 1 to 5, wherein the amivantamab is administered intravenously (IV) at a dose of about 1050 mg.
Embodiment 7. The method of any one of Embodiments 1 to 5, wherein the amivantamab is administered intravenously (IV) at a dose of about 1400 mg.
Embodiment 8. The method of Embodiments 6 or 7, wherein the dose is administered weekly for 4 weeks, followed by administration every two weeks.
Embodiment 8a. The method of Embodiment 8, wherein the dose is administered until disease progression or unacceptable toxicity or complete response.
Embodiment 9. The method of Embodiment 8, wherein the dose administered during the first week is a split dose.
Embodiment 9a. The method of Embodiment 9, wherein the split dose is administered in two or more infusions over a period of no more than 3 days.
Embodiment 10. A method of selling an approved drug product comprising amivantamab, the method comprising selling such drug product, wherein a drug product label for a reference listed drug for such drug product includes instructions for treating NSCLC.
Embodiment 11. The method of Embodiment 10, wherein the drug product is a biosimilar drug product, a Biologic License Application drug product or a supplemental Biologic License Application drug product.
Embodiment 12. A method of offering for sale an approved drug product comprising amivantamab, the method comprising offering for sale such drug product, wherein a drug product label for a reference listed drug for such drug product includes instructions for treating NSCLC.
Embodiment 13. The method of Embodiment 12, wherein the drug product is a biosimilar drug product, a Biologic License Application drug product or a supplemental Biologic License Application drug product.
Embodiment 14. A method of selling an approved drug product comprising amivantamab, the method comprising selling such drug product, wherein the drug product label for a reference listed drug for such drug product comprises overall response rate (ORR) data according to Response Evaluation Criteria in Solid Tumors (RECIST v1.1).
Embodiment 15. A method of offering for sale an approved drug product comprising amivantamab, said method comprising offering for sale such drug product, wherein the drug product label for a reference listed drug for such drug product comprises overall response rate (ORR) data according to Response Evaluation Criteria in Solid Tumors (RECIST v1.1).
Embodiment 16. A method of improving overall response in an adult patient with NSCLC, wherein the overall response is measured according to Response Evaluation Criteria in Solid Tumors (RECIST v1.1), the method comprising administering to the adult patient an approved drug product comprising amivantamab.
Embodiment 17. The method of embodiment 14, 15 or 16, wherein the approved drug product is a biosimilar drug product, a Biologic License Application drug product or a supplemental Biologic License Application drug product.
Embodiment 18. The method of embodiment 16, wherein the NSCLC is locally advanced or metastatic.
Embodiment 19. The method of embodiment 16, wherein the NSCLC has an at least one epidermal growth factor receptor (EGFR) exon 20 insertion mutation.
Embodiment 20. The method of embodiment 16, wherein the patient was previously administered a platinum-based chemotherapy drug.
Embodiment 20a. The method of embodiment 20 wherein the platinum-based chemotherapy drug is selected from the group consisting of cisplatin, carboplatin, oxaliplatin, nedaplatin, lobaplatin and heptaplatin.
Embodiment 21 The method of embodiment 16, wherein the approved drug product provides an improvement in the overall response, wherein the overall response is either complete response (CR) or partial response (PR) as measured according to RECIST v1.1.
Embodiment 22. The method of embodiment 16, wherein a drug product label for a reference listed drug for such approved drug product comprises overall response rate (ORR) data according to Response Evaluation Criteria in Solid Tumors (RECIST v1.1).
Embodiment 23. The method of embodiment 16, wherein a drug product label for a reference listed drug for such approved drug product includes instructions for treating NSCLC.
Embodiment 24. The method of embodiment 16, further comprising selling such approved drug product, wherein a drug product label for a reference listed drug for such approved drug product includes instructions for treating NSCLC.
Embodiment 25. The method of embodiment 24, wherein the drug product label comprises overall response rate (ORR) data.
Embodiment 26. A method of improving overall response in an adult patient with NSCLC, said method comprising providing to said patient an approved drug product comprising amivantamab.
Embodiment 27. The method of embodiment 26, wherein the NSCLC is locally advanced or metastatic.
Embodiment 28. The method of embodiment 26, wherein the NSCLC has an at least one epidermal growth factor receptor (EGFR) exon 20 insertion mutation.
Embodiment 29. The method of embodiment 26, wherein the patient was previously administered a platinum-based chemotherapy drug.
Embodiment 30. The method of embodiment 26, wherein a drug product label for a reference listed drug for such approved drug product comprises overall response rate (ORR) data.
Embodiment 31. The method of embodiment 26, wherein a drug product label for a reference listed drug for such approved drug product includes instructions for treating NSCLC.

### Example 1: FDA-Approved Prescribing Information for RYBREVANT™

### Full Prescribing Information

### 1 Indications and Usage

RYBREVANT is indicated for the treatment of adult patients with locally advanced or metastatic non-small cell lung cancer (NSCLC) with epidermal growth factor receptor (EGFR) exon 20 insertion mutations, as detected by an FDA-approved test [see Dosage and Administration (2.1)], whose disease has progressed on or after platinum-based chemotherapy.

This indication is approved under accelerated approval based on overall response rate and duration of response [see Clinical Studies (14)]. Continued approval for this indication may be contingent upon verification and description of clinical benefit in the confirmatory trials.

### 2 Dosage and Administration

### 2.1 Patient Selection

Select patients for treatment with RYBREVANT based on the presence of EGFR exon 20 insertion mutations [see Clinical Studies (14.1)]. Information on FDA-approved tests is available at: http://www.fda.gov/CompanionDiagnostics.

### 2.2 Recommended Dosage

The recommended doses of RYBREVANT, based on baseline body weight, are provided in Table 1. Administer RYBREVANT weekly for 4 weeks, with the initial dose as a split infusion in Week 1 on Day 1 and Day 2, then administer every 2 weeks thereafter until disease progression or unacceptable toxicity. Administer premedications before each RYBREVANT infusion as recommended [see Dosage and Administration (2.3)]. Administer diluted RYBREVANT intravenously according to the infusion rates in Table 5 [see Dosage and Administration (2.5), (2.6)].

TABLE 1
Recommended Dose of RYBREVANT Based on Baseline Body Weight

| Body Weight | Recommended | Number of 350 mg/7 mL |
|---|---|---|
| at Baseline* | Dose | RYBREVANT Vials |
| Less than 80 kg | 1050 mg | 3 |
| Greater than or | 1400 mg | 4 |
| equal to 80 kg | | |

*Dose adjustments not required for subsequent body weight changes.

### 2.3 Recommended Premedications

Prior to initial infusion of RYBREVANT (Week 1, Days 1 and 2), administer premedication as described in Table 2 to reduce the risk of infusion-related reactions: [see Warnings and Precautions (5.1)]

TABLE 2
Premedications

| | | | Dosing Window |
|---|---|---|---|
| | | | Prior to |
| | | Route of | RYBREVANT |
| Medication | Dose | Administration | Administration |
| Antihistamine* | Diphenhydramine | Intravenous | 15 to |
| | (25 to 50 mg) or | | 30 minutes |
| | equivalent | Oral | 30 to |
| | | | 60 minutes |
| Antipyretic* | Acetaminophen | Intravenous | 15 to |
| | (650 to 1,000 mg) | | 30 minutes |
| | | Oral | 30 to |
| | | | 60 minutes |
| Glucocorticoid^{‡} | Dexamethasone | Intravenous | 45 to |
| | (10 mg) or | | 60 minutes |
| | Methylprednisolone | | |
| | (40 mg) or equivalent | | |

*Required at all doses.
^{‡}Required at in initial dose (Week 1, Days 1 and 2); optional for subsequent doses.

Administer both antihistamine and antipyretic prior to all infusions. Glucocorticoid administration required for Week 1, Days 1 and 2 doses only and as necessary for subsequent infusions.

### 2.4 Dosage Modifications for Adverse Reactions

The recommended RYBREVANT dose reductions for adverse reactions (see Table 4) are listed in Table 3.

TABLE 3
RYBREVANT Dose Reductions for Adverse Reactions

| Body Weight | Initial | 1^{st} Dose | 2^{nd} Dose | 3^{rd} Dose |
|---|---|---|---|---|
| at Baseline | Dose | Reduction | Reduction | Reduction |
| Less than 80 kg | 1050 mg | 700 mg | 350 mg | Discontinue |
| Greater than or | 1400 mg | 1050 mg | 700 mg | RYBREVANT |
| equal to 80 kg | | | | |

The recommended RYBREVANT dosage modifications for adverse reactions are provided in Table 4.

TABLE 4
Recommended RYBREVANT Dosage Modifications for Adverse Reactions

| Adverse Reaction | Severity | Dosage Modifications |
|---|---|---|
| Infusion-related reactions | Grade 1 to 2 | Interrupt RYBREVANT infusion if IRR is |
| (IRR) [see Warnings and | | suspected and monitor patient until reaction |
| Precautions (5.1)] | | symptoms resolve. |
| | | Resume the infusion at 50% of the infusion rate |
| | | at which the reaction occurred. |
| | | If there are no additional symptoms after |
| | | 30 minutes, the infusion rate may be escalated |
| | | (see Table 5). |
| | | Include corticosteroid with-premedications for |
| | | subsequent dose (see Table 2). |
| | Grade 3 | Interrupt RYBREVANT infusion and |
| | | administer supportive care medications. |
| | | Monitor patient until reaction symptoms |
| | | resolve. |
| | | Resume the infusion at 50% of the infusion rate |
| | | at which the reaction occurred. |
| | | If there are no additional symptoms after |
| | | 30 minutes, the infusion rate may be escalated |
| | | (see Table 5). |
| | | Include corticosteroid with premedications for |
| | | subsequent dose (see Table 2). For recurrent |
| | | Grade 3, permanently discontinue |
| | | RYBREVANT. |
| | Grade 4 | Permanently discontinue RYBREVANT. |
| Interstitial Lung Disease | Any Grade | Withhold RYBREVANT if ILD/pneumonitis is |
| (ILD)/pneumonitis [see | | suspected. |
| Warnings and Precautions | | Permanently discontinue RYBREVANT if |
| (5.2)]. | | ILD/pneumonitis is confirmed. |
| Dermatologic Adverse | Grade 2 | Initiate supportive care management. |
| Reactions (including | | Reassess after 2 weeks; if rash does not |
| dermatitis acneiform, | | improve, consider dose reduction. |
| pruritus, dry skin) [see | Grade 3 | Withhold RYBREVANT and initiate supportive |
| Warnings and Precautions | | care management. |
| (5.3)] | | Upon recovery to ≤ Grade 2, resume |
| | | RYBREVANT at reduced dose. |
| | | If no improvement within 2 weeks, permanently |
| | | discontinue treatment. |
| | Grade 4 | Permanently discontinue RYBREVANT |
| | Severe bullous, | Permanently discontinue RYBREVANT. |
| | blistering or | |
| | exfoliating skin | |
| | conditions | |
| | (including toxic | |
| | epidermal | |
| | necrolysis (TEN) | |
| Other Adverse Reactions | Grade 3 | Withhold RYBREVANT until recovery to ≤ |
| [see Adverse Reactions | | Grade 1 or baseline. |
| (6.1)] | | Resume at the same dose if recovery occurs |
| | | within 1 week. |
| | | Resume at reduced dose if recovery occurs after |
| | | 1 week but within 4 weeks. |
| | | Permanently discontinue if recovery does not |
| | | occur within 4 weeks. |
| | Grade 4 | Withhold RYBREVANT until recovery to ≤ |
| | | Grade 1 or baseline. |
| | | Resume at reduced dose if recovery occurs |
| | | within 4 weeks. |
| | | Permanently discontinue if recovery does not |
| | | occur within 4 weeks. |
| | | Permanently discontinue for recurrent Grade 4 |
| | | reactions. |

### 2.5 Preparation

Dilute and prepare RYBREVANT for intravenous infusion before administration.
Check that the RYBREVANT solution is colorless to pale yellow. Parenteral drug products should be inspected visually for particulate matter and discoloration prior to administration, whenever solution and container permit. Do not use if discoloration or visible particles are present.
Determine the dose required (either 1050 mg or 1400 mg) and number of RYBREVANT vials needed based on patient's baseline weight [see Dosage and Administration (2.2)]. Each vial of RYBREVANT contains 350 mg of amivantamab-vmjw.
Withdraw and then discard a volume of either 5% dextrose solution or 0.9% sodium chloride solution from the 250 mL infusion bag equal to the volume of RYBREVANT to be added (i.e., discard 7 mL diluent from the infusion bag for each RYBREVANT vial). Only use infusion bags made of polyvinylchloride (PVC), polypropylene (PP), polyethylene (PE), or polyolefin blend (PP+PE).
Withdraw 7 mL of RYBREVANT from each vial and add it to the infusion bag. The final volume in the infusion bag should be 250 mL. Discard any unused portion left in the vial.
Gently invert the bag to mix the solution. Do not shake.
Diluted solutions should be administered within 10 hours (including infusion time) at room temperature 59° F. to 77° F. (15° C. to 25° C.).

### 2.6 Administration

Administer the diluted solution [see Dosage and Administration (2.5)] by intravenous infusion using an infusion set fitted with a flow regulator and with an in-line, sterile, non-pyrogenic, low protein-binding polyethersulfone (PES) filter (pore size 0.2 micrometer) primed with diluent only. Administration sets must be made of either polyurethane (PU), polybutadiene (PBD) PVC, PP, or PE.

Do not infuse RYBREVANT concomitantly in the same intravenous line with other agents.

Administer RYBREVANT via a peripheral line on Week 1 and Week 2 given the high incidence of infusion-related reactions during initial treatment [see Warnings and Precautions (5.1)]. RYBREVANT may be administered via central line for subsequent weeks. For the initial infusion, prepare RYBREVANT as close to administration time as possible to allow for the possibility of extended infusion time in the event of an infusion-related reaction.

Administer RYBREVANT infusion intravenously according to the infusion rates in Table 5.

TABLE 5
Infusion Rates for RYBREVANT Administration

| | | | |
|---|---|---|---|
| | Dose | Initial | Subsequent |
| Week | (per 250 mL bag) | Infusion Rate | Infusion Rate^{†} |

1050 mg Dose

| | | | |
|---|---|---|---|
| Week 1 (split dose infusion) | | | |
| Week 1 Day 1 | 350 mg | 50 mL/hr | 75 mL/hr |
| Week 1 Day 2 | 700 mg | 50 mL/hr | 75 mL/hr |

| | | |
|---|---|---|
| Week 2 | 1050 mg | 85 mL/hr |
| Week 3 | 1050 mg | 125 mL/hr |
| Week 4 | 1050 mg | 125 mL/hr |
| Subsequent weeks* | 1050 mg | 125 mL/hr |

1400 mg Dose

| | | | |
|---|---|---|---|
| Week 1 (split dose infusion) | | | |
| Week 1 Day 1 | 350 mg | 50 mL/hr | 75 mL/hr |
| Week 1 Day 2 | 1050 mg | 35 mL/hr | 50 mL/hr |
| Week 2 | 1400 mg | 65 mL/hr | |

| | | |
|---|---|---|
| Week 3 | 1400 mg | 85 mL/hr |
| Week 4 | 1400 mg | 125 mL/hr |
| Subsequent weeks* | 1400 mg | 125 mL/hr |

*After Week 4, patients are dosed every 2 weeks.
^{†}Increase the initial infusion rate to the subsequent infusion rate after 2 hours in the absence of infusion-related reactions.

### 3 Dosage Forms and Strengths

Injection: 350 mg/7 mL (50 mg/mL) colorless to pale yellow solution in a single-dose vial.

### 4 Contraindications

None.

### 5 Warnings and Precautions

### 5.1 Infusion-Related Reactions

RYBREVANT can cause infusion-related reactions (IRR); signs and symptoms of IRR include dyspnea, flushing, fever, chills, nausea, chest discomfort, hypotension, and vomiting.

Based on the safety population [see Adverse Reactions (6.1)], IRR occurred in 66% of patients treated with RYBREVANT. Among patients receiving treatment on Week 1 Day 1, 65% experienced an IRR, while the incidence of IRR was 3.4% with the Day 2 infusion, 0.4% with the Week 2 infusion, and cumulatively 1.1% with subsequent infusions. Of the reported IRRs, 97% were Grade 1-2, 2.2% were Grade 3, and 0.4% were Grade 4. The median time to onset was 1 hour (range 0.1 to 18 hours) after start of infusion. The incidence of infusion modifications due to IRR was 62% and 1.3% of patients permanently discontinued RYBREVANT due to IRR.

Premedicate with antihistamines, antipyretics, and glucocorticoids and infuse RYBREVANT as recommended [see Dosage and Administration (2.3)]. Administer RYBREVANT via a peripheral line on Week 1 and Week 2 [see Dosage and Administration (2.6)].

Monitor patients for any signs and symptoms of infusion reactions during RYBREVANT infusion in a setting where cardiopulmonary resuscitation medication and equipment are available. Interrupt infusion if IRR is suspected. Reduce the infusion rate or permanently discontinue RYBREVANT based on severity [see Dosage and Administration (2.4)].

### 5.2 Interstitial Lung Disease/Pneumonitis

RYBREVANT can cause interstitial lung disease (ILD)/pneumonitis. Based on the safety population [see Adverse Reactions (6.1)], ILD/pneumonitis occurred in 3.3% of patients treated with RYBREVANT, with 0.7% of patients experiencing Grade 3 ILD/pneumonitis. Three patients (1%) discontinued RYBREVANT due to ILD/pneumonitis.

Monitor patients for new or worsening symptoms indicative of ILD/pneumonitis (e.g., dyspnea, cough, fever). Immediately withhold RYBREVANT in patients with suspected ILD/pneumonitis and permanently discontinue if ILD/pneumonitis is confirmed [see Dosage and Administration (2.4)].

### 5.3 Dermatologic Adverse Reactions

RYBREVANT can cause rash (including dermatitis acneiform), pruritus and dry skin. Based on the safety population [see Adverse Reactions (6.1)], rash occurred in 74% of patients treated with RYBREVANT, including Grade 3 rash in 3.3% of patients. The median time to onset of rash was 14 days (range: 1 to 276 days). Rash leading to dose reduction occurred in 5% of patients, and RYBREVANT was permanently discontinued due to rash in 0.7% of patients [see Adverse Reactions (6.1)].

Toxic epidermal necrolysis (TEN) occurred in one patient (0.3%) treated with RYBREVANT.

Instruct patients to limit sun exposure during and for 2 months after treatment with RYBREVANT. Advise patients to wear protective clothing and use broad-spectrum UVA/UVB sunscreen. Alcohol-free emollient cream is recommended for dry skin.

If skin reactions develop, start topical corticosteroids and topical and/or oral antibiotics. For Grade 3 reactions, add oral steroids and consider dermatologic consultation. Promptly refer patients presenting with severe rash, atypical appearance or distribution, or lack of improvement within 2 weeks to a dermatologist. Withhold, dose reduce or permanently discontinue RYBREVANT based on severity [see Dosage and Administration (2.4)].

### 5.4 Ocular Toxicity

RYBREVANT can cause ocular toxicity including keratitis, dry eye symptoms, conjunctival redness, blurred vision, visual impairment, ocular itching, and uveitis. Based on the safety population [see Adverse Reactions (6.1)], keratitis occurred in 0.7% and uveitis occurred in 0.3% of patients treated with RYBREVANT. All events were Grade 1-2. Promptly refer patients presenting with eye symptoms to an ophthalmologist. Withhold, dose reduce or permanently discontinue RYBREVANT based on severity [see Dosage and Administration (2.4)].

### 5.5 Embryo-Fetal Toxicity

Based on its mechanism of action and findings from animal models, RYBREVANT can cause fetal harm when administered to a pregnant woman. Administration of other EGFR inhibitor molecules to pregnant animals has resulted in an increased incidence of impairment of embryo-fetal development, embryolethality, and abortion. Advise females of reproductive potential of the potential risk to the fetus. Advise female patients of reproductive potential to use effective contraception during treatment and for 3 months after the final dose of RYBREVANT. [see Use in Specific Populations (8.1, 8.3)].

### 6 Adverse Reactions

The following adverse reactions are discussed elsewhere in the labeling:
Infusion-Related Reactions [see Warnings and Precautions (5.1)]
Interstitial Lung Disease/Pneumonitis [see Warnings and Precautions (5.2)]
Dermatologic Adverse Reactions [see Warnings and Precautions (5.3)]
Ocular Toxicity [see Warnings and Precautions (5.4)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety population described in the WARNINGS AND PRECAUTIONS reflect exposure to RYBREVANT as a single agent in the CHRYSALIS study in 302 patients with locally advanced or metastatic NSCLC who received a dose of 1050 mg (for patients <80 kg) or 1400 mg (for patients ≥80 kg) once weekly for 4 weeks, then every 2 weeks thereafter. Among 302 patients who received RYBREVANT, 36% were exposed for 6 months or longer and 12% were exposed for greater than one year. In the safety population, the most common (≥20%) adverse reactions were rash, infusion-related reaction, paronychia, musculoskeletal pain, dyspnea, nausea, edema, cough, fatigue, stomatitis, constipation, vomiting and pruritus. The most common Grade 3 to 4 laboratory abnormalities (≥2%) were decreased lymphocytes, decreased phosphate, decreased albumin, increased glucose, increased gamma glutamyl transferase, decreased sodium, decreased potassium, and increased alkaline phosphatase.

The data described below reflect exposure to RYBREVANT at the recommended dosage in 129 patients with locally advanced or metastatic NSCLC with EGFR exon 20 insertion mutations whose disease had progressed on or after platinum-based chemotherapy. Among patients who received RYBREVANT, 44% were exposed for 6 months or longer and 12% were exposed for greater than one year.

The median age was 62 years (range: 36 to 84 years); 61% were female; 55% were Asian, 35% were White, and 2.3% were Black; and 82% had baseline body weight <80 kg.

Serious adverse reactions occurred in 30% of patients who received RYBREVANT. Serious adverse reactions in ≥2% of patients included pulmonary embolism, pneumonitis/ILD, dyspnea, musculoskeletal pain, pneumonia, and muscular weakness. Fatal adverse reactions occurred in 2 patients (1.5%) due to pneumonia and 1 patient (0.8%) due to sudden death.

Permanent discontinuation of RYBREVANT due to an adverse reaction occurred in 11% of patients. Adverse reactions resulting in permanent discontinuation of RYBREVANT in ≥1% of patients were pneumonia, IRR, pneumonitis/ILD, dyspnea, pleural effusion, and rash.

Dose interruptions of RYBREVANT due to an adverse reaction occurred in 78% of patients. Infusion-related reactions (IRR) requiring infusion interruptions occurred in 59% of patients. Adverse reactions requiring dose interruption in ≥5% of patients included dyspnea, nausea, rash, vomiting, fatigue, and diarrhea.

Dose reductions of RYBREVANT due to an adverse reaction occurred in 15% of patients. Adverse reactions requiring dose reductions in ≥2% of patients included rash and paronychia.

The most common adverse reactions (≥20%) were rash, IRR, paronychia, musculoskeletal pain, dyspnea, nausea, fatigue, edema, stomatitis, cough, constipation, and vomiting. The most common Grade 3 to 4 laboratory abnormalities (≥2%) were decreased lymphocytes, decreased albumin, decreased phosphate, decreased potassium, increased glucose, increased alkaline phosphatase, increased gamma-glutamyl transferase, and decreased sodium.

Table 6 summarizes the adverse reactions in CHRYSALIS.

TABLE 6
Adverse Reactions (≥10%) in Patients with NSCLC with Exon 20 Insertion
Mutations Whose Disease Has Progressed on or after Platinum-based Chemotherapy
and Received RYBREVANT in CHRYSALIS

| | RYBREVANT | |
|---|---|---|
| | (N = 129) | |
| Adverse Reactions | All Grades (%) | Grades 3 or 4 (%) |

Skin and subcutaneous tissue disorders

| | | |
|---|---|---|
| Rash^{a} | 84 | 3.9 |
| Pruritus | 18 | 0 |
| Dry skin | 14 | 0 |

General disorders and administration site conditions

| | | |
|---|---|---|
| Infusion related reaction | 64 | 3.1 |
| Fatigue^{b} | 33 | 2.3 |
| Edema^{c} | 27 | 0.8 |
| Pyrexia | 13 | 0 |

Infections and infestations

| | | |
|---|---|---|
| Paronychia | 50 | 3.1 |
| Pneumonia^{d} | 10 | 0.8 |

Musculoskeletal and connective tissue disorders

| | | |
|---|---|---|
| Musculoskeletal pain^{e} | 47 | 0 |

Respiratory, thoracic and mediastinal disorders

| | | |
|---|---|---|
| Dyspnea^{f} | 37 | 2.3 |
| Cough^{g} | 25 | 0 |

Gastrointestinal disorders

| | | |
|---|---|---|
| Nausea | 36 | 0 |
| Stomatitis^{h} | 26 | 0.8 |
| Constipation | 23 | 0 |
| Vomiting | 22 | 0 |
| Diarrhea | 16 | 3.1 |
| Abdominal Painⁱ | 11 | 0.8 |

Vascular disorders

| | | |
|---|---|---|
| Hemorrhage^{j} | 19 | 0 |

Metabolism and nutrition disorders

| | | |
|---|---|---|
| Decreased appetite | 15 | 0 |

Nervous system disorders

| | | |
|---|---|---|
| Peripheral neuropathy^{k} | 13 | 0 |
| Dizziness | 12 | 0.8 |
| Headache^{l} | 10 | 0.8 |

^{a}Rash: acne, dermatitis, dermatitis acneiform, eczema, eczema asteatotic, palmar-plantar erythrodysesthesia syndrome, perineal rash, rash, rash erythematous, rash maculo-papular, rash papular, rash vesicular, skin exfoliation, toxic epidermal necrolysis
^{b}Fatigue: asthenia, fatigue
^{c}Edema: eyelid edema, face edema, generalized edema, lip edema, edema, edema peripheral, periorbital edema, peripheral swelling
^{d}Pneumonia: atypical pneumonia, lower respiratory tract infection, pneumonia, pneumonia aspiration, and pulmonary sepsis
^{e}Musculoskeletal pain: arthralgia, arthritis, back pain, bone pain, musculoskeletal chest pain, musculoskeletal discomfort, musculoskeletal pain, myalgia, neck pain, non-cardiac chest pain, pain in extremity, spinal pain
^{f} Dyspnea: dyspnea, dyspnea exertional
^{g}Cough: cough, productive cough, upper airway cough syndrome
^{h}Stomatitis: aphthous ulcer, cheilitis, glossitis, mouth ulceration, mucosal inflammation, pharyngeal inflammation, stomatitis
ⁱAbdominal pain: abdominal discomfort, abdominal pain, abdominal pain lower, abdominal pain upper, and epigastric discomfort
^{j}Hemorrhage: epistaxis, gingival bleeding, hematuria, hemoptysis, hemorrhage, mouth hemorrhage, mucosal hemorrhage
^{k}Peripheral neuropathy: hypoesthesia, neuralgia, paresthesia, peripheral sensory neuropathy
^{l}Headache: headache, migraine

Clinically relevant adverse reactions in <10% of patients who received RYBREVANT included ocular toxicity, ILD/pneumonitis, and toxic epidermal necrolysis (TEN).

Table 7 summarizes the laboratory abnormalities in CHRYSALIS.

TABLE 7
Select Laboratory Abnormalities (≥20%) That Worsened from Baseline
in Patients With Metastatic NSCLC with EGFR Exon 20 Insertion
Mutations Whose Disease Has Progressed on or After Platinum-based
Chemotherapy and Who Received RYBREVANT in CHRYSALIS

| | RYBREVANT⁺ | |
|---|---|---|
| | (N = 129) | |
| | All Grades | Grades 3 or 4 |
| Laboratory Abnormality | (%) | (%) |
| Chemistry | | |
| Decreased albumin | 79 | 8 |
| Increased glucose | 56 | 4 |
| Increased alkaline phosphatase | 53 | 4.8 |
| Increased creatinine | 46 | 0 |
| Increased alanine aminotransferase | 38 | 1.6 |
| Decreased phosphate | 33 | 8 |
| Increased aspartate aminotransferase | 33 | 0 |
| Decreased magnesium | 27 | 0 |
| Increased gamma-glutamyl transferase | 27 | 4 |
| Decreased sodium | 27 | 4 |
| Decreased potassium | 26 | 6 |
| Hematology | | |
| Decreased lymphocytes | 36 | 8 |

⁺The denominator used to calculate the rate was 126 based on the number of patients with a baseline value and at least one post-treatment value.

### 6.2 Immunogenicity

As with all therapeutic proteins, there is the potential for immunogenicity. The detection of antibody formation is highly dependent on the sensitivity and specificity of the assay. Additionally, the observed incidence of antibody (including neutralizing antibody) positivity in an assay may be influenced by several factors including assay methodology, sample handling, timing of sample collection, concomitant medications, and underlying disease. For these reasons, comparison of the incidence of antibodies in the studies described below with the incidence of antibodies in other studies or to other amivantamab products may be misleading.

In CHRYSALIS, 3 of the 286 (1%) patients who were treated with RYBREVANT and evaluable for the presence of anti-drug antibodies (ADA), tested positive for treatment-emergent anti-amivantamab-vmjw antibodies (one at 27 days, one at 59 days and one at 168 days after the first dose) with titers of 1:40 or less. There are insufficient data to evaluate the effect of ADA on the pharmacokinetics, safety, or efficacy of RYBREVANT.

### 8 Use in Specific Populations

### 8.1 Pregnancy

### Risk Summary

Based on the mechanism of action and findings in animal models, RYBREVANT can cause fetal harm when administered to a pregnant woman. There are no available data on the use of RYBREVANT in pregnant women or animal data to assess the risk of RYBREVANT in pregnancy. Disruption or depletion of EGFR in animal models resulted in impairment of embryo-fetal development including effects on placental, lung, cardiac, skin, and neural development. The absence of EGFR or MET signaling has resulted in embryolethality, malformations, and post-natal death in animals (see Data). Advise pregnant women of the potential risk to a fetus.

In the U.S. general population, the estimated background risk of major birth defects and miscarriage in clinically recognized pregnancies is 2% to 4% and 15% to 20%, respectively.

### Data

### Animal Data

No animal studies have been conducted to evaluate the effects of amivantamab-vmjw on reproduction and fetal development; however, based on its mechanism of action, RYBREVANT can cause fetal harm or developmental anomalies. In mice, EGFR is critically important in reproductive and developmental processes including blastocyst implantation, placental development, and embryo-fetal/postnatal survival and development. Reduction or elimination of embryo-fetal or maternal EGFR signaling can prevent implantation, can cause embryo-fetal loss during various stages of gestation (through effects on placental development) and can cause developmental anomalies and early death in surviving fetuses. Adverse developmental outcomes were observed in multiple organs in embryos/neonates of mice with disrupted EGFR signaling. Similarly, knock out of MET or its ligand HGF was embryonic lethal due to severe defects in placental development, and fetuses displayed defects in muscle development in multiple organs. Human IgG1 is known to cross the placenta; therefore, amivantamab-vmjw has the potential to be transmitted from the mother to the developing fetus.

### 8.2 Lactation

### Risk Summary

There are no data on the presence of amivantamab-vmjw in human milk on milk production, or its effects on the breastfed child. Because of the potential for serious adverse reactions from RYBREVANT in breast-fed infants, advise women not to breast-feed during treatment with RYBREVANT and for 3 months after the final dose.

### 8.3 Females and Males of Reproductive Potential

RYBREVANT can cause fetal harm when administered to a pregnant woman [see Use in Specific Populations (8.1)].

### Pregnancy Testing

Verify pregnancy status of females of reproductive potential prior to initiating RYBREVANT.

### Contraception

### Females

Advise females of reproductive potential to use effective contraception during treatment and for 3 months after the final dose of RYBREVANT.

### 8.4 Pediatric Use

The safety and efficacy of RYBREVANT have not been established in pediatric patients.

### 8.5 Geriatric Use

Of the 129 patients treated with RYBREVANT, 41% were 65 years of age or older, and 9% were 75 years of age or older. No clinically important differences in safety or efficacy were observed between patients who were ≥65 years of age and younger patients.

### 11 Description

Amivantamab-vmjw is a low-fucose human immunoglobulin G1-based bispecific antibody directed against the EGF and MET receptors, produced by mammalian cell line (Chinese Hamster Ovary [CHO]) using recombinant DNA technology that has a molecular weight of approximately 148 kDa. RYBREVANT (amivantamab-vmjw) injection for intravenous infusion is a sterile, preservative-free, colorless to pale yellow solution in single-dose vials. The pH is 5.7.

Each RYBREVANT vial contains 350 mg (50 mg/mL) amivantamab-vmjw, EDTA disodium salt dihydrate (0.14 mg), L-histidine (2.3 mg), L-histidine hydrochloride monohydrate (8.6 mg), L-methionine (7 mg), polysorbate 80 (4.2 mg), sucrose (595 mg), and water for injection, USP.

### 12 Clinical Pharmacology

### 12.1 Mechanism of Action

Amivantamab-vmjw is a bispecific antibody that binds to the extracellular domains of EGFR and MET.

In in vitro and in vivo studies amivantamab-vmjw was able to disrupt EGFR and MET signaling functions through blocking ligand binding and, in exon 20 insertion mutation models, degradation of EGFR and MET. The presence of EGFR and MET on the surface of tumor cells also allows for targeting of these cells for destruction by immune effector cells, such as natural killer cells and macrophages, through antibody-dependent cellular cytotoxicity (ADCC) and trogocytosis mechanisms, respectively.

### 12.2 Pharmacodynamics

The exposure-response relationship and time-course of pharmacodynamic response of amivantamab-vmjw have not been fully characterized in patients with NSCLC with EGFR exon 20 insertion mutations.

### 12.3 Pharmacokinetics

Amivantamab-vmjw exposures increased proportionally over a dosage range from 350 to 1750 mg (0.25 to 1.25 times the maximum approved recommended dosage). Steady state of amivantamab-vmjw concentrations was achieved by the 9th infusion. The accumulation ratio at steady state was 2.4.

### Distribution

The amivantamab-vmjw mean (±SD) volume of distribution is 5.13 (±1.78) L.

### Elimination

The mean (±SD) clearance of amivantamab-vmjw is 360 (±144) mL/day and the terminal half-life is 11.3 (±4.53) days.

### Specific Populations

No clinically meaningful differences in the pharmacokinetics of amivantamab-vmjw were observed based on age (range: 32-87 years), sex, race, creatinine clearance (CLcr 29 to 276 mL/min), or mild hepatic impairment [(total bilirubin≤ULN and AST>ULN) or (ULN<total bilirubin≤1.5 times ULN)]. The pharmacokinetics of amivantamab-vmjw have not been studied in patients with severe renal impairment (CLcr 15 to 29 mL/min) or patients with moderate (total bilirubin 1.5 to 3 times ULN) to severe (total bilirubin>3 times ULN) hepatic impairment.

### Body Weight

Increases in body weight increased the volume of distribution and clearance of amivantamab-vmjw. Amivantamab-vmjw exposures are 30-40% lower in patients who weighed ≥80 kg compared to patients with body weight <80 kg at the same dose. Exposures of amivantamab-vmjw were comparable between patients who weighed <80 kg and received 1050 mg dose and patients who weighed ≥80 kg and received 1400 mg dose.

### 13 Nonclinical Toxicology

### 13.1 Carcinogenesis, Mutagenesis, Impairment of Fertility

No studies have been performed to assess the potential of amivantamab-vmjw for carcinogenicity or genotoxicity. Fertility studies have not been performed to evaluate the potential effects of amivantamab-vmjw. In 6-week and 3-month repeat-dose toxicology studies in monkeys, there were no notable effects in the male and female reproductive organs.

### 14 Clinical Studies

The efficacy of RYBREVANT was evaluated in patients with locally advanced or metastatic NSCLC with EGFR exon 20 insertion mutations in a multicenter, open-label, multi-cohort clinical trial (CHRYSALIS, NCT02609776). The study included patients with locally advanced or metastatic NSCLC with EGFR exon 20 insertion mutations whose disease had progressed on or after platinum-based chemotherapy. Patients with untreated brain metastases and patients with a history of ILD requiring treatment with prolonged steroids or other immunosuppressive agents within the last 2 years were not eligible for the study.

In the efficacy population, EGFR exon 20 insertion mutation status was determined by prospective local testing using tissue (94%) and/or plasma (6%) samples. Of the 81 patients with EGFR exon 20 insertion mutations, plasma samples from 96% of patients were tested retrospectively using Guardant360® CDx. While 76% of patients had an EGFR exon 20 insertion mutation identified in plasma specimen, 20% did not have an EGFR exon 20 insertion mutation identified in plasma specimen, and 3.7% did not have plasma samples for testing.

Patients received RYBREVANT at 1050 mg (for patient baseline body weight <80 kg) or 1400 mg (for patient baseline body weight ≥80 kg) once weekly for 4 weeks, then every 2 weeks thereafter until disease progression or unacceptable toxicity. The major efficacy outcome measure was overall response rate (ORR) according to Response Evaluation Criteria in Solid Tumors (RECIST v1.1) as evaluated by Blinded Independent Central Review (BICR). An additional efficacy outcome measure was duration of response (DOR) by BICR.

The efficacy population included 81 patients with NSCLC with EGFR exon 20 insertion mutation with measurable disease who were previously treated with platinum-based chemotherapy. The median age was 62 (range: 42 to 84) years, 59% were female; 49% were Asian, 37% were White, 2.5% were Black; 74% had baseline body weight <80 kg; 95% had adenocarcinoma; and 46% had received prior immunotherapy. The median number of prior therapies was 2 (range: 1 to 7). At baseline, 67% had Eastern Cooperative Oncology Group (ECOG) performance status of 1; 53% never smoked; all patients had metastatic disease; and 22% had previously treated brain metastases.

Efficacy results are summarized in Table 8.

TABLE 8
Efficacy Results for CHRYSALIS

| | Prior Platinum-based |
|---|---|
| | Chemotherapy Treated |
| | (N = 81) |
| Overall Response Rate (95% CI) | 40% (29%, 51%) |
| Complete response (CR) | 3.7% |
| Partial response (PR) | 36% |
| Duration of Response (DOR) | |
| Median, months (95% CI), months | 11.1 (6.9, NE) |
| Patients with DOR ≥6 months | 63% |

Based on Kaplan-Meier estimates.
NE = Not Estimable, CI = confidence interval.

### 16 how Supplied/Storage and Handling

### How Supplied

RYBREVANT™ (amivantamab-vmjw) injection is a sterile, preservative-free, colorless to pale yellow solution for intravenous infusion. Each single-dose vial contains 350 mg/7 mL (50 mg/mL) RYBREVANT. Each vial is individually packed in a single carton. (NDC 57894-501-01).

### Storage and Handling

Store in a refrigerator at 2° C. to 8° C. (36° F. to 46° F.) in original carton to protect from light. Do not freeze.

### 17 Patient Counseling Information

Advise the patient to read the FDA-approved patient labeling (Patient Information).

### Infusion-Related Reactions

Advise patients that RYBREVANT can cause infusion-related reactions, the majority of which may occur with the first infusion. Advise patients to alert their healthcare provider immediately for any signs or symptoms of infusion-related reactions [see Warnings and Precautions (5.1)].

### Interstitial Lung Disease/Pneumonitis

Advise patients of the risks of interstitial lung disease (ILD)/pneumonitis. Advise patients to immediately contact their healthcare provider for new or worsening respiratory symptoms [see Warnings and Precautions (5.2)].

### Dermatologic Adverse Reactions

Advise patients of the risk of dermatologic adverse reactions. Advise patients to limit direct sun exposure, to use broad spectrum UVA/UVB sunscreen, and to wear protective clothing during treatment with RYBREVANT [see Warnings and Precautions (5.3)]. Advise patients to apply alcohol free emollient cream to dry skin.

### Ocular Toxicity

Advise patients of the risk of ocular toxicity. Advise patients to contact their ophthalmologist if they develop eye symptoms and advise discontinuation of contact lenses until symptoms are evaluated [see Warnings and Precautions (5.4)].

### Paronychia

Advise patients of the risk of paronychia. Advise patients to contact their healthcare provider for signs or symptoms of paronychia [see Adverse Reactions (6.1)].

### Embryo-Fetal Toxicity

Advise females of reproductive potential of the potential risk to a fetus, to use effective contraception during treatment with RYBREVANT and for 3 months after the final dose, and to inform their healthcare provider of a known or suspected pregnancy. [see Warnings and Precautions (5.5), Use in Specific Populations (8.1, 8.3)].

### Lactation

Advise women not to breastfeed during treatment with RYBREVANT and for 3 months after the final dose [see Use in Specific Populations (8.2)].

### Product of Ireland

### Manufactured by:

### Janssen Biotech, Inc.

### Horsham, Pa. 19044

### U.S. License Number 1864

We claim:

1. A method of treating non-small cell lung cancer (NSCLC) comprising administering an approved drug product comprising amivantamab to an adult patient with NSCLC in an amount that is described in a drug product label for the drug product.

2. The method of claim 1, wherein the NSCLC is locally advanced or metastatic.

3. The method of claim 1, wherein the NSCLC has an at least one epidermal growth factor receptor (EGFR) exon 20 insertion mutation.

4. The method of claim 1, wherein the patient was previously administered a platinum based chemotherapy drug.

5. The method of claim 1, wherein administration of the approved product comprising amivantamab provides an improvement in overall response, wherein the overall response is measured according to Response Evaluation Criteria in Solid Tumors (RECIST v1.1) of the adult patient with NSCLC.

6. The method of claim 1, wherein the amivantamab is administered intravenously (IV) at a dose of about 1050 mg.

7. The method of claim 1, wherein the amivantamab is administered intravenously (IV) at a dose of about 1400 mg.

8. The method of claim 6 or 7, wherein the dose is administered weekly for 4 weeks, followed by administration every two weeks.

9. The method of claim 8, wherein the dose administered during the first week is a split dose.

10. A method of selling an approved drug product comprising amivantamab, the method comprising selling such drug product, wherein a drug product label for a reference listed drug for such drug product includes instructions for treating NSCLC.

11. The method of claim 10, wherein the drug product is a biosimilar drug product, a Biologic License Application drug product or a supplemental Biologic License Application drug product.

12. A method of offering for sale an approved drug product comprising amivantamab, the method comprising offering for sale such drug product, wherein a drug product label for a reference listed drug for such drug product includes instructions for treating NSCLC.

13. The method of claim 12, wherein the drug product is a biosimilar drug product, a Biologic License Application drug product or a supplemental Biologic License Application drug product.

14. A method of selling an approved drug product comprising amivantamab, the method comprising selling such drug product, wherein the drug product label for a reference listed drug for such drug product comprises overall response rate (ORR) data according to Response Evaluation Criteria in Solid Tumors (RECIST v1.1).

15. A method of offering for sale an approved drug product comprising amivantamab, said method comprising offering for sale such drug product, wherein the drug product label for a reference listed drug for such drug product comprises overall response rate (ORR) data according to Response Evaluation Criteria in Solid Tumors (RECIST v1.1).

16. A method of improving overall response in an adult patient with NSCLC, wherein the overall response is measured according to Response Evaluation Criteria in Solid Tumors (RECIST v1.1), the method comprising administering to the adult patient an approved drug product comprising amivantamab.

17. The method of claim 14, 15 or 16, wherein the approved drug product is a biosimilar drug product, a Biologic License Application drug product or a supplemental Biologic License Application drug product.

18. The method of claim 16, wherein the NSCLC is locally advanced or metastatic.

19. The method of claim 16, wherein the NSCLC has an at least one epidermal growth factor receptor (EGFR) exon 20 insertion mutation.

20. The method of claim 16, wherein the patient was previously administered a platinum-based chemotherapy drug.

21. The method of claim 16, wherein the approved drug product provides an improvement in the overall response, wherein the overall response is either complete response (CR) or partial response (PR) as measured according to RECIST v1.1.

22. The method of claim 16, wherein a drug product label for a reference listed drug for such approved drug product comprises overall response rate (ORR) data according to Response Evaluation Criteria in Solid Tumors (RECIST v1.1).

23. The method of claim 16, wherein a drug product label for a reference listed drug for such approved drug product includes instructions for treating NSCLC.

24. The method of claim 16, further comprising selling such approved drug product, wherein a drug product label for a reference listed drug for such approved drug product includes instructions for treating NSCLC.

25. The method of claim 24, wherein the drug product label comprises overall response rate (ORR) data.

26. A method of improving overall response in an adult patient with NSCLC, said method comprising providing to said patient an approved drug product comprising amivantamab.

27. The method of claim 26, wherein the NSCLC is locally advanced or metastatic.

28. The method of claim 26, wherein the NSCLC has an at least one epidermal growth factor receptor (EGFR) exon 20 insertion mutation.

29. The method of claim 26, wherein the patient was previously administered a platinum-based chemotherapy drug.

30. The method of claim 26, wherein a drug product label for a reference listed drug for such approved drug product comprises overall response rate (ORR) data.

31. The method of claim 26, wherein a drug product label for a reference listed drug for such approved drug product includes instructions for treating NSCLC.


SEQUENCE LISTING (22 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 1210 aa, Homo sapiens [sequence omitted]

SEQ ID NO: 22 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS